(19) United States
(12) Patent Application Publication
(10) Pub. No.: US 2025/0258260 A1
(43) Pub. Date: Aug. 14, 2025

(54) MEGLUMINE BASED FORMULATIONS FOR DYNAMIC NUCLEAR POLARIZATION

(71) Applicant: THE BOARD OF REGENTS OF THE UNIVERSITY OF TEXAS SYSTEM, Austin, TX (US)

(72) Inventors: Zoltan Kovacs, Lewisville, TX (US); Jae Mo Park, Coppell, TX (US); Eul Hyun Suh, Murphy, TX (US); Sara Chirayil, Plano, TX (US); Mai Huynh, Garland, TX (US)

(21) Appl. No.: 19/050,737

(22) Filed: Feb. 11, 2025

Related U.S. Application Data
(60) Provisional application No. 63/552,386, filed on Feb. 12, 2024.

(51) Int. Cl. G01R 33/44

(52) U.S. Cl. CPC G01R 33/445

(57) ABSTRACT

Provided herein are formulations including a carboxylic acid, a glassing solvent, and a contrast agent. The formulation may further include an aqueous medium and a chelating agent or a surfactant. The formulation may be polarized. Also provided herein are methods of making and using the polarized formulations.

### CROSS-REFERENCE TO RELATED APPLICATION

This application claims the benefit of U.S. Provisional Application Ser. No. 63/552,386 filed Feb. 12, 2024, of which is hereby incorporated by reference in its entirety.

### FIELD

The present disclosure relates to sugar alcohol-based formulations for hyperpolarized (HP) nuclear magnetic resonance (NMR)/magnetic resonance imaging (MRI). Also provided herewith are methods of making and using the sugar alcohol-based formulations.

### BACKGROUND

NMR techniques may be applied to gather information about a sample or sample area in a gentle, non-destructive way; in particular, clinical investigations on living patients can be done non-invasively. However, NMR techniques are generally limited by low signal intensities.

One way to increase signal intensities is to apply hyperpolarization techniques. In some examples, nuclei in a sample are prepared with a polarization level higher than corresponding to the Boltzmann distribution at the sample's temperature, and the hyperpolarized nuclei undergo an NMR experiment.

HP NMR/MRI is a collective term for various technologies developed to improve the sensitivity of standard NMR/MRI. These include techniques that operate on physics principles such as dynamic nuclear polarization (DNP) and spin exchange optical pumping as well as the chemistry-based approaches such as parahydrogen induced polarization. Hyperpolarization technology dramatically widens the scope of metabolic MRI by enabling the real-time in vivo detection of non-proton nuclei that are difficult to measure otherwise (e.g., ¹³C, ¹⁵N, and ²⁹Si).

Dissolution DNP is a widely used method to enhance the sensitivity of liquid-state MR signals largely because it is generally applicable to all MR active nuclei and can increase the MR signal by several orders of magnitude.

Experimentally, the compound to be polarized is dissolved in a glass forming solvent or mixture of solvents and doped with a small amount of paramagnetic substance, usually a stable free radical. The microwave driven DNP is performed by irradiating this sample near the Electron Paramagnetic Resonance (EPR) frequency at low temperature and in high magnetic field. The solid-state polarization build-up is monitored and when it reaches plateau the sample is rapidly dissolved with superheated solvent.

Dissolution DNP works by transferring the high electron spin polarization to coupled nuclear spins at cryogenic temperatures followed by rapid dissolution of the frozen DNP sample. The most prominent applications of dissolution DNP are to polarize ¹³C-labeled metabolic substrates for in vivo C NMR spectroscopy and imaging. In vivo HP C magnetic resonance spectroscopic imaging (MRSI) can provide read-outs of metabolite levels, pH, or redox state or can be used to monitor enzyme-catalyzed biochemical processes in real-time, as they happen. A commonly used HP agent is ¹³C-labeled pyruvate because it polarizes extremely well by DNP, quickly transported into cells and lies at a key intersection in intermediary metabolism where it can be oxidized to acetyl-CoA+CO₂ via pyruvate dehydrogenase (PDH) in mitochondria, forms alanine via transamination, or exchanges with tissue lactate via lactate dehydrogenase. Translational studies with HP [1-¹³C]pyruvate are actively being performed.

### SUMMARY

In an aspect, the current disclosure encompasses a formulation comprising an amino alcohol and a substrate. The amino alcohol may include an amino alcohol salt. The substrate comprises one or more of a free or ester of an organic acid. The organic acid comprises C₃-C₆ monocarboxylic acid, C₃-C₆ dicarboxylic acid, C₃-C₆ hydroxy acid, C₃-C₆ α-keto-acid, amino acid, or a combination thereof. The formulation may be an imaging agent.

In another aspect, the current disclosure encompasses a method of manufacturing a formulation for DNP-NMR. The method comprises combining a substrate with meglumine to form a glassing matrix.

In yet another aspect, the current disclosure encompasses a method of imaging a sample. The method comprises exposing a formulation to the sample and conducting dynamic nuclear polarization (DNP)-nuclear magnetic resonance (NMR) on the sample after exposure to the formulation.

### BRIEF DESCRIPTION OF THE DRAWINGS

FIG. 1A shows the steps in the process of making a polarized formulation in some examples of the present disclosure.

FIG. 1B illustrates an example of a polarizer used to make a polarized formulation in some examples of the present disclosure.

FIG. 2A illustrates the chemical reaction of fumaric acid and meglumine to form meglumine fumarate salt in some examples of the present disclosure.

FIG. 2B is a pictorial representation 3.6 M of meglumine-fumarate (left) and fumaric acid in DMSO (right) at room temperature after 90 minutes sonication in some examples of the present disclosure.

FIG. 2C is a pictorial representation of 3.6 M of meglumine fumarate (left) and fumaric acid in DMSO (right) after freezing in liquid nitrogen in some examples of the present disclosure.

FIG. 2D is a pictorial representation of 3.6 M of meglumine-fumarate (left) and fumaric acid in DMSO (right) after thawing to the room temperature in some examples of the present disclosure.

FIG. 2E is a pictorial representation of 3.6 M of meglumine-fumarate (left) and fumaric acid in DMSO (right) after thawing to the room temperature and sonicating 40 minutes at 40° C. in some examples of the present disclosure.

FIG. 2F is a pictorial representation of 4.8 M of meglumine-fumarate (left) and fumaric acid in DMSO (right) at room temperature after 90 minutes sonication in some examples of the present disclosure.

FIG. 3A shows a time-resolved HP ¹³C normalized NMR spectra of meglumine [1,4-¹³C₂]fumarate in some examples of the present disclosure.

FIG. 3B shows a time-resolved HP ¹³C normalized NMR spectra of DMSO [1,4-¹³C₂]fumarate in some examples of the present disclosure.

FIG. 3C shows a representative first time-resolved HP ¹³C normalized NMR spectra of meglumine [1,4-¹³C₂]fumarate in some examples of the present disclosure.

FIG. 3D shows a representative first time-resolved HP ¹³C normalized NMR spectra of DMSO [1,4-¹³C₂]fumarate in some examples of the present disclosure.

FIG. 3E shows a representative relaxation of the HP meglumine [1,4-¹³C₂]fumarate in some examples of the present disclosure.

FIG. 3F shows a representative ¹³C polarization of meglumine [1,4-¹³C₂]fumarate and DMSO [1,4-¹³C₂]fumarate in some examples of the present disclosure.

FIGS. 4A-D show performance comparison of meglumine [1,4-¹³C₂]fumarate and DMSO [1,4-¹³C₂]fumarate in healthy rat kidneys in vivo.

FIGS. 5A-F show in vivo detection of mitochondrial products in healthy rat kidneys using HP meglumine [1,4-¹³C₂]fumarate.

### DETAILED DESCRIPTION

The following detailed description references the accompanying drawings that illustrate various aspects of the present disclosure. The drawings and description are intended to describe aspects and aspects of the present disclosure in sufficient detail to enable those skilled in the art to practice the present disclosure. Other components can be utilized, and changes can be made without departing from the scope of the present disclosure. The following description is, therefore, not to be taken in a limiting sense.

The DNP process requires an isotropic glassing matrix, in which the molecules are randomly oriented. Therefore, the composition and vitrification properties of the frozen sample is an important consideration. The DNP sample must be a homogenous mixture and is generally composed of the substrate to be polarized, the polarizing agent and, if necessary, glassing agents. DNP polarizers are designed to accommodate a few hundred μL of a sample volume. The SPINlab polarizer, the only available clinical DNP polarizer at this time, may polarize up to ˜2 mL of sample. For human studies, the concentration of the HP substrate in the administered solution is around 250 mM. This means that the concentration of the substrate in the DNP sample must be on the order of 1 M to 10 M to produce an injectable solution of the HP substrate. Some compounds such as pyruvic acid form a glass at cryogenic temperatures without adding any vitrifying agents. However, a majority of substrates need to be dissolved in a glassing matrix for DNP. Commonly used biocompatible matrix for in vivo work is a mixture of glycerol and water, but other glassing matrices such as dimethyl sulfoxide (DMSO) are also used. While the disclosure discusses DMSO, DMSO-glycerol can also be utilized.

Fumarate may require a glassing matrix such as conventional DMSO for DNP. With conventional DMSO, fumaric acid concentration may reach 3.6 M. However, due to its extremely low solubility in water (6 g/L at 25° C.), HP fumaric acid may quickly precipitate after dissolution, limiting the final concentration of fumarate in injectate. Moreover, although conventional fumaric acid DMSO solutions will form a glass upon rapid cooling to cryogenic temperatures, the acid can crystalize when the sample is warmed after an initial rapid freezing process, as demonstrated in this study. This creates a practical problem of using conventional DMSO-fumaric acid mixtures for DNP.

The present disclosure is directed to a new formulation for dissolution DNP. The present disclosure is also directed to a method of making the new formulation. Lastly, the present disclosure is also directed to a method of using the formulation.

The present disclosure relates to a formulation may include amino alcohol and a substrate. The amino alcohol may include meglumine. In some examples, the amino alcohol can include an amino alcohol salt. In some examples, the amino alcohol salt can include a salt of meglumine. The substrate may include one or more of a free or ester form of an organic acid. The formulation may include an aqueous medium. The formulation further may include a contrast agent.

The formulation may be an imaging agent. The imaging agent may be used for dynamic nuclear polarization (DNP)-nuclear magnetic resonance (NMR). The combination of the amino alcohol and organic acid may improve the solubility of the organic acid in water resulting in a transparent solution at room temperature and a high concentration of the imaging agent. In some examples, the imaging agent has a concentration of at least about 3 M to about 5 M in an aqueous medium. In at least one example, the imaging agent has a concentration of at least 4.8 M in an aqueous medium.

The organic acid may include C₃-C₆ monocarboxylic acid, C₃-C₆ dicarboxylic acid, C₃-C₆ hydroxy acid, C₃-C₆ α-keto-acid, amino acid, or a combination thereof. In some examples, the C₃-C₆ hydroxy acid may include C₃-C₆ α-hydroxy acid, C₃-C₆ β-hydroxy acid, C₄-C₆ γ-hydroxy acid, or a combination thereof. In some examples, the polarizing agent and/or the contrast agent may include a paramagnetic species such as free radicals, metal ions, nano particles or a combination of these, such as trityl OXO63, EPA, nitroxide radicals, gadolinium salts, and complexes. In some examples, the monocarboxylic acid may include butyric acid and the salt or ester is butyrate. In some examples, the dicarboxylic acid may include fumaric acid, succinic acid, malic acid, oxaloacetic acid, or a combination thereof and the salt or ester is fumarate, succinate, maleate, oxaloacetate, or a combination thereof. In some examples, the hydroxy acid may include lactic acid and the salt or ester is lactate, β-hydroxy butyric acid, malic acid, or a combination thereof. In some examples, the α-keto acid may include pyruvic acid and the salt or ester may include pyruvate, oxaloacetic acid, α-ketoglutaric acid, or a combination thereof.

The present disclosure further relates to a method of manufacturing a formulation for DNP-NMR. The method may include combining a substrate with meglumine to form a glassing matrix. In at least one example, the method can include loading the glassing matrix to a polarizer using a multistep lowering procedure (e.g., 14 steps). The glassing matrix compound for DNP may be loaded to the polarizer using a procedure that includes more than 2 steps. In some examples, the glassing matrix compound can be loaded to the polarizer using a multistep lowering procedure that includes between 10 and 20 steps. The method may include adding a dissolution media to the glassing matrix compound to form the formulation.

In some examples, the dissolution media includes water with 0.1 g/L ethylenediaminetetraacetic acid (EDTA). In some examples, the glassing matrix compound for DNP is loaded to the polarizer using the 14 steps lowering procedure. In some examples, the substrate includes fumarate, pyruvate, butyrate, amino acids, and/or lactate. In some examples, the glassing matrix includes polarizing agent and/or contrast agent such as trityl OXO63, EPA, nitroxide radicals, gadolinium salts, and complexes. In some examples, the glassing matrix can include OXO63.

The present disclosure further relates to a method of manufacturing the disclosed formulation for DNP-NMR. The method comprises combining a substrate with meglumine to form a glassing matrix. In some examples, the substrate includes fumarate, pyruvate, butyrate, amino acids, and/or lactate. In some examples, the glassing matrix includes the polarizing agent and/or contrast agent which may be a paramagnetic species such as free radicals, metal ions, nano particles, and/or a combination thereof, such as trityl OXO63, EPA, nitroxide radicals, gadolinium salts, and/or complexes. The method further comprises loading the glassing matrix compound for DNP-NMR into the polarizer using the multistep lowering procedure. Given the ability of the formulation to avoid crystallization, the glassing matrix compound can be incrementally loaded to the polarizer using the multistep lower procedure (e.g., more than 2 steps) without crystallization. Accordingly, polarizer does not receive an excessive heat load from the formulation. As such, the glassing matrix compound for DNP-NMR may be incrementally loaded into the polarizer using the multistep lowering procedure which can include more than 2 steps. In some examples, the glassing matrix compound for DNP-NMR is loaded into the polarizer using the multistep lowering procedure which can include 14 steps. In some examples, the glassing matrix compound for DNP-NMR may be incrementally loaded into the polarizer using the multistep lowering procedure which can include between 10 and 25 steps. The presence of a sugar alcohol like meglumine may result in a high concentration of imaging agent in an aqueous medium because of the high solubility of the sugar alcohol. In addition, no crystallization is observed in formulations with a sugar alcohol.

The method comprises adding a dissolution media to the polarized glassing matrix compound. In some examples, the dissolution media includes water with 0.1 g/L ethylenediaminetetraacetic acid (EDTA).

The present disclosure further relates to a method of imaging a sample. The method comprises exposing the disclosed formulation to the sample and conducting dynamic nuclear polarization (DNP)-nuclear magnetic resonance (NMR) on the sample after exposure to the disclosed formulation.

### I. Formulation

The present disclosure is directed to a formulation for dissolution DNP-NMR. The formulation includes a substrate. The formulation may include a solvent. The solvent may be an aqueous medium. The formulation may include a contrast agent. The formulation may be polarized resulting in a polarized formulation. The formulation may be an imaging agent.

The substrate includes an organic acid. In at least one example, the substrate may include an amino alcohol as a counter ion. The substrate can form an amino alcohol salt and one or more of a free or ester form of an organic acid.

The amino alcohol salt and one or more of a free or ester form of an organic acid may be formed by an amino alcohol and an organic acid.

In some examples, the amino alcohol may include meglumine. Meglumine is a sugar alcohol derived from glucose that contains an amino group modification. Meglumine is a non-toxic and non-metabolizable derivative of sorbitol with a strong structural similarity to glycerol. Meglumine may be often used as an excipient in pharmaceuticals and in conjunction with iodinated compounds in contrast media such as diatrizoate meglumine, iothalamate meglumine, and iodipamide meglumine. It may also be used with some Gd-based contrast agents. The solubility of meglumine in an aqueous medium may be up to about 1 g/mL. Due to the extremely high solubility in water (up to 1 g/mL), meglumine can increase the aqueous solubility of the formulation. The high solubility of meglumine in polar protic solvents is the result of intermolecular hydrogen bonding between the meglumine and the solvent molecules.

In some examples, the organic acid comprises monocarboxylic acid, dicarboxylic acid, hydroxy acid, keto acid, amino acid, or a combination thereof. The monocarboxylic acid may include two or more carbons. For example, the monocarboxylic acid may have two, three, four, five, six, seven, or eight carbon atoms. In some examples, the monocarboxylic acid may have three, four, five, or six carbon atoms. The dicarboxylic acid may include two or more carbons. For example, the dicarboxylic acid may have two, three, four, five, six, seven, or eight carbon atoms. In some examples, the dicarboxylic acid may have three, four, five, or six carbon atoms. The hydroxy acid includes α-hydroxy acid, β-hydroxy acid, or γ-hydroxy acid. The hydroxy acid may include two or more carbons. For example, the hydroxy acid may have two, three, four, five, six, seven, or eight carbon atoms. In some examples, the hydroxy acid may have three, four, five, or six carbon atoms. In some examples, the hydroxy acid may be C₃-C₆ α-hydroxy acid. In some examples, the hydroxy acid may be C₃-C₆ β-hydroxy acid. In some examples, the hydroxy acid may be C₄-C₆ γ-hydroxy acid. The keto acid includes α-keto acid, β-keto acid, or γ-keto acid. The keto acid may include two or more carbons. For example, the keto acid may have two, three, four, five, six, seven, or eight carbon atoms. In some examples, the keto acid may have three, four, five, or six carbon atoms. In some examples, the keto acid may be C₃-C₆ α-hydroxy acid. In some examples, the keto acid may be C₃-C₆ β-hydroxy acid. In some examples, the keto acid may be C₄-C₆ γ-hydroxy acid.

In some examples, the C₃-C₆ monocarboxylic acid may be butyric acid and the salt or ester may be butyrate. In some examples, the dicarboxylic acid comprises fumaric acid, succinic acid, malic acid, oxaloacetic acid, or a combination thereof and the salt or ester is fumarate, succinate, maleate, oxaloacetate, or a combination thereof. In some examples, the C₃-C₆ α-hydroxy acid may be lactic acid and the salt or ester is lactate. In some examples, the hydroxy acid may be β-hydroxy butyric acid. In some examples, the hydroxy acid may be a hydroxy acid and a dicarboxy acid including but not limited to α-hydroxy dicarboxylic acid.

In some examples, the dicarboxylic acid comprises fumaric acid, succinic acid, malic acid, oxaloacetic acid, or a combination thereof and the salt or ester is fumarate, succinate, maleate, oxaloacetate, or a combination thereof.

In some examples, the α-keto acid may be pyruvic acid and the salt or ester may be pyruvate. In some examples, the α-keto acid may include keto-dicarboxylic acids including but not limited to α-ketoglutaric acid.

In some examples, the contrast agent may be a substantially transparent solution at room temperature. In some examples, the contrast agent may include the polarizing agent or contrast agent which may be a paramagnetic species such as free radicals, metal ions, nano particles, and/or a combination thereof, such as trityl OXO63, EPA, nitroxide radicals, gadolinium salts, and/or complexes.

In some examples, the aqueous medium may be water.

The concentration of the amino alcohol and the organic acid may be from about 2 M to about 6 M in the aqueous medium. For example, the concentration of the imaging agent may be about 2 M, about 2.1 M, about 2.2 M, about 2.3 M, about 2.4 M, about 2.5 M, about 2.6 M, about 2.7 M, about 2.8 M, about 2.9 M, about 3.0 M, about 3.1 M, about 3.2 M, about 3.3 M, about 3.4 M, about 3.5 M, about 3.6 M, about 3.7 M, about 3.8 M, about 3.9 M, about 4.0 M, about 4.1 M, about 4.2 M, about 4.3 M, about 4.4 M, about 4.5 M, about 4.6 M, about 4.7 M, about 4.8 M, about 4.9 M, about 5.0 M. In some examples, the concentration of the imaging agent in the aqueous medium may be about 4.8 M.

The concentration of the contrast agent in the aqueous medium may range from about 5 mM to about 50 mM. For example, the concentration may be about 5 mM, about 6 mM, about 7 mM, about 8 mM, about 9 mM, about 10 mM, about 11 mM, about 12 mM, about 13 mM, about 14 mM, 15 mM, about 16 mM, about 17 mM, about 18 mM, about 19 mM, about 20 mM, about 21 mM, about 22 mM, about 23 mM, about 24 mM, 25 mM, about 26 mM, about 27 mM, about 28 mM, about 29 mM, about 30 mM, about 31 mM, about 32 mM, about 33 mM, about 34 mM, 35 mM, about 36 mM, about 37 mM, about 38 mM, about 39 mM, about 40 mM, about 41 mM, about 42 mM, about 43 mM, about 44 mM, 45 mM, about 46 mM, about 47 mM, about 48 mM, about 49 mM, or about 50 mM. In some examples, the concentration of the contrast agent may be 15 mM.

In some examples, the substrate may include but in not limited to fumarate, pyruvate, butyrate, amino acids, or a combination thereof. In some examples, the substrate may be meglumine [1,4-¹³C₂]fumarate.

Fumarate may require a glassing matrix such as DMSO in conventional formulations and glycerol for DNP. With conventional DMSO, fumaric acid concentration may reach 3.6 M. However, due to its extremely low solubility in water (6 g/L at 25° C.), conventional HP fumaric acid may quickly precipitate after dissolution, limiting the final concentration of fumarate in injectate. Moreover, although fumaric acid DMSO solutions form a glass upon rapid cooling to cryogenic temperatures, the acid can crystalize when the sample is warmed after an initial rapid freezing process. This creates a practical problem of using conventional DMSO-fumaric acid mixtures for DNP, as the crystallization is caused by the polarization of the DMSO-fumaric acid mixture. As the mixture has been polarized, the imaging resolution is greatly and negatively impacted.

Here, meglumine [1,4-¹³C₂]fumarate may be prepared by neutralizing fumaric acid with meglumine. Meglumine as a counterion significantly improved the solubility and glassing properties of fumarate, which may result in increased ¹³C polarization. Meglumine is a non-toxic and non-metabolizable derivative of sorbitol with a strong structural similarity to glycerol. Due to the extremely high solubility in water (for example up to 1 g/mL), meglumine increases the aqueous solubility of the formulation disclosed herein. The high solubility of meglumine in polar protic solvents is the result of intermolecular hydrogen bonding between the meglumine and the solvent molecules. Meglumine [1,4-¹³C₂]fumarate can be prepared by neutralizing fumaric acid with meglumine.

The disclosed formulation may have excellent vitrification properties. For example, the disclosed formulation may be stored at −80° C. for more than 3 months without crystallization.

### II. Method of Making a Formulation

In at least one example, the current disclosure encompasses a method of making the formulation. The formulation may include an imaging agent. The imaging agent may be used for dissolution DNP-NMR.

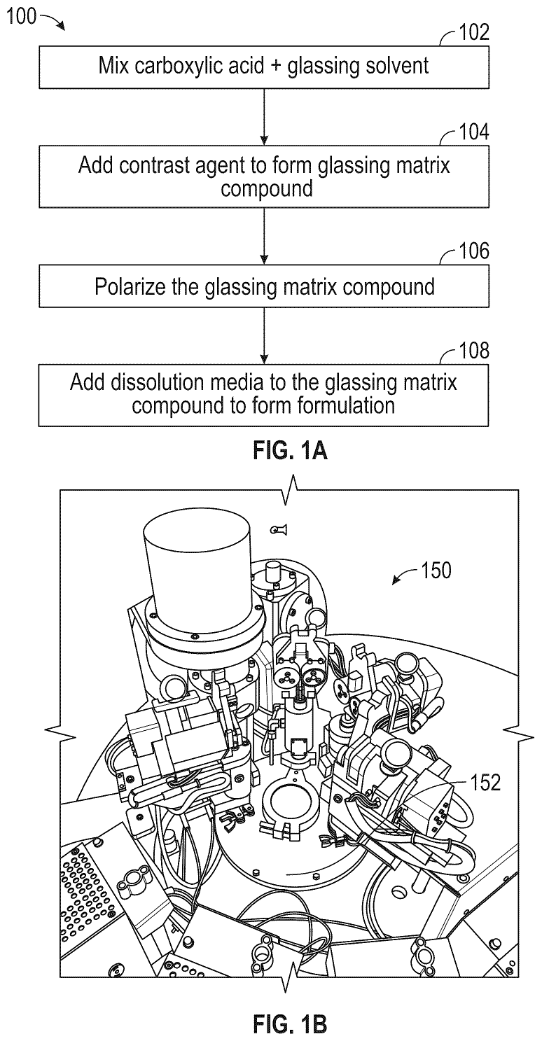

Referring to FIG. 1A, a flowchart is presented in accordance with an example embodiment. The method 100 is provided by way of example, as there are a variety of ways to carry out the method. The method 100 described below can be carried out using the configurations illustrated in FIGS. 1B-5F, for example, and various elements of these figures are referenced in explaining example method 100. Each block shown in FIG. 1A represents one or more processes, methods or subroutines, carried out in the example method 100. Furthermore, the illustrated order of blocks is illustrative only and the order of the blocks can change according to the present disclosure. Additional blocks may be added, or fewer blocks may be utilized, without departing from this disclosure.

The method 100 can begin at block 102. At block 102, the method includes combining a substrate with a solvent to form a glassing matrix. The substrate may include a carboxylic acid. The substrate may further include fumarate, pyruvate, butyrate, amino acids, and/or lactate. At block 104, the method includes adding a contrast agent to the glassing matrix to form a glassing matrix compound. The contrast agent may include the polarizing agent and/or contrast agent which may be a paramagnetic species such as free radicals, metal ions, nano particles, and/or a combination thereof, such as trityl OXO63, EPA, nitroxide radicals, gadolinium salts, and/or complexes. At block 106, the method includes polarizing the glassing matrix compound to form a polarized matrix. For example, as shown in FIG. 1B, a polarizer 150 can be operable to receive the glassing matrix compound. The glassing matrix compound can be loaded into the polarizer at loading portion 152. The glassing matrix compound can be loaded into the polarizer using a multistep lowering procedure that can include a plurality of steps. In at least one example, the glassing matrix compound may be incrementally loaded into the polarizer using the multistep lowering procedure which can include more than 2 steps. In some examples, the glassing matrix compound may be incrementally loaded into the polarizer using the multistep lowering procedure which can include more than 14 steps. In some examples, the glassing matrix compound may be incrementally loaded into the polarizer using the multistep lowering procedure which can include less than 20 steps. In some examples, the glassing matrix compound may be incrementally loaded into the polarizer using the multistep lowering procedure which can include less than 25 steps. At block 108, the method includes adding a dissolution media to the polarized glassing matrix compound to form the formulation.

In some examples, the organic acid comprises monocarboxylic acid, dicarboxylic acid, hydroxy acid, keto acid, amino acid, or any combination thereof. The monocarboxylic acid may include two or more carbons. For example, the monocarboxylic acid may have two, three, four, five, six, seven, or eight carbon atoms. In some examples, the monocarboxylic acid may have three, four, five, or six carbon atoms. The dicarboxylic acid may include two or more carbons. For example, the dicarboxylic acid may have two, three, four, five, six, seven, or eight carbon atoms. In some examples, the dicarboxylic acid may have three, four, five, or six carbon atoms. The hydroxy acid includes α-hydroxy acid, β-hydroxy acid, or γ-hydroxy acid. The hydroxy acid may include two or more carbons. For example, the hydroxy acid may have two, three, four, five, six, seven, or eight carbon atoms. In some examples, the hydroxy acid may have three, four, five, or six carbon atoms. In some examples, the hydroxy acid may be C₃-C₆ α-hydroxy acid. In some examples, the hydroxy acid may be C₃-C₆ β-hydroxy acid. In some examples, the hydroxy acid may be C₄-C₆ γ-hydroxy acid. The keto acid includes α-keto acid, β-keto acid, or γ-keto acid. The keto acid may include two or more carbons. For example, the keto acid may have two, three, four, five, six, seven, or eight carbon atoms. In some examples, the keto acid may have three, four, five, or six carbon atoms. In some examples, the keto acid may be C₃-C₆ α-hydroxy acid. In some examples, the keto acid may be C₃-C₆ β-hydroxy acid. In some examples, the keto acid may be C₄-C₆ γ-hydroxy acid.

In some examples, the C₃-C₆ monocarboxylic acid may include butyric acid and the salt or ester may be butyrate. In some examples, the dicarboxylic acid comprises fumaric acid, succinic acid, malic acid, oxaloacetic acid, or any combination thereof and the salt or ester is fumarate, succinate, maleate, oxaloacetate, or any combination thereof. In some examples, the C₃-C₆ α-hydroxy acid may include lactic acid and the salt or ester include lactate. In some examples, the hydroxy acid may include β-hydroxy butyric acid. In some examples, the hydroxy acid may include both a hydroxy acid and a dicarboxy acid including but not limited to α-hydroxy dicarboxylic acid.

In some examples, the dicarboxylic acid comprises fumaric acid, succinic acid, malic acid, oxaloacetic acid, or any combination thereof and the salt or ester is fumarate, succinate, maleate, oxaloacetate, or any combination thereof.

In some examples, the α-keto acid may be pyruvic acid and the salt or ester may include pyruvate. In some examples, the α-keto acid may include keto-dicarboxylic acids including but not limited to α-ketoglutaric acid.

In some examples, the contrast agent may be a substantially transparent solution at room temperature. In some examples, the contrast agent may include the polarizing agent and/or contrast agent which may be a paramagnetic species such as free radicals, metal ions, nano particles, and/or a combination thereof, such as trityl OXO63, EPA, nitroxide radicals, gadolinium salts, and/or complexes.

In some examples, the glassing matrix with the contrast agent (e.g., the glassing matrix compound) may be loaded incrementally into the polarizer using the multistep lowering procedure that includes more than 2 steps, more than 3 steps, more than 4 steps, more than 5 steps, more than 10 steps, more than 20 steps, more than 25 steps, or more than 30 steps. In some examples, the sample (e.g., the glassing matrix compound) may be incrementally loaded into the polarizer using the multistep lowering procedure that includes 11 steps, 12 steps, 13 steps, 14 steps, 15 steps, 16 steps, 17 steps, 18 steps, 19 steps, or 20 steps.

In some examples, the sugar alcohol may include meglumine.

The dissolution media may be an aqueous solution. The dissolution media may further include a chelating agent, a surfactant, or any combination thereof. Examples of suitable chelating agents and/or surfactants include, but are not limited to, ethylenediaminetetraacetic acid (EDTA), dimercaptosuccinic acid, picolinic acid, ethylenebis(oxyethylenenitrilo)tetraacetic acid (EGTA), 1,2-cyclohexylenedinitrilotetraacetic acid (CDTA), diethylenetriaminepentaacetic acid (DTPA), or any combination thereof. In some examples, the chelating agent and/or surfactant may be ethylenediaminetetraacetic acid (EDTA).

The concentration of the chelating agent and/or surfactant may range from about 0.05 g/mL to about 1 g/mL. For example, the concentration may be about 0.05 g/mL, about 0.06 g/mL, about 0.07 g/mL, about 0.08 g/mL, about 0.09 g/mL, about 0.1 g/mL, about 0.2 g/mL, about 0.3 g/mL, about 0.4 g/mL, about 0.5 g/mL, about 0.6 g/mL, about 0.7 g/mL, about 0.8 g/mL, about 0.9 g/mL, or about 1 g/mL. In some examples, the concentration of EDTA may be about 0.1 g/mL.

The formulation of the present disclosure may include fumarate, pyruvate, butyrate, amino acids, and/or lactate.

In some examples, meglumine fumarate may be prepared by mixing one equivalent of fumaric acid with two equivalents of meglumine free base in the presence of trace amount of water (approximately 6% by weight) followed by sonication for 90 minutes. pH of resulting preparation may range from about 7.0 to about 8.0. For example, the pH may be about 7.0, about 7.1, about 7.2, about 7.3, about 7.4, about 7.5, about 7.6, about 7.7, about 7.8, about 7.9, or about 8.0.

Meglumine fumarate may vitrify by itself in the presence of trace amounts of water. The formulations may be polarized in the clinical SPINlab polarizer. Notably, conventional fumaric acid DMSO samples may be loaded in the SPINlab using the fast-lowering procedure to avoid crystallization of fumaric acid. This single step lowering process may not be practical for large samples (e.g., human dose) for creating excessive heat load to the polarizer. However, the superior glassing properties of meglumine fumarate may overcome this limitation and these samples may be loaded in the polarizer using the standard multistep lowering procedure. Fumarate concentrations in the formulations may be about 4.8 M, which may result in 120 mM HP fumarate solutions after dissolution. In addition, since the meglumine fumarate samples may be prepared by neutralizing fumaric acid with two equivalents of meglumine, further neutralization during the dissolution may not be necessary. On the other hand, conventional DMSO-fumaric acid samples must be neutralized with neutralizing media (0.72 M of NaOH, 0.4 M TRIS, and 0.4 g/L EDTA).

The disclosed formulations are simple to prepare without crystallization compared to formulations prepared using conventional DMSO-fumaric acid mixture. For example, to make the disclosed formulations a neutralization step is not needed.

### III. Method of Imaging a Sample

This disclosure is further related to a method of imaging a sample. The method includes exposing the disclosed formulation to the sample of interest and conducting dynamic nuclear polarization (DNP)-nuclear magnetic resonance (NMR) on the sample of interest after exposure to the formulation.

Clinical translation of many hyperpolarized substrates, including ¹³C-fumarate, has been obstructed by its low solubility in water, causing rapid precipitation of the substrates after dissolution when prepared in conventional glassing solvents such as glycerol and DMSO. As previously mentioned, the use of meglumine dramatically enhances water solubility and glassing properties of fumarate. [¹³C]bicarbonate images captured using hyperpolarized meglumine [1,4-¹³C₂]fumarate may demonstrate unique potentials for investigating gluconeogenic pathways in vivo, particularly in metabolic syndromes such as type 2 diabetes and chronic kidney diseases, which develop altered gluconeogenesis.

HP ¹³C-labeled fumarate may be a promising probe for clinical translation. The appearance of HP [1,4-¹³C₂]malate from HP [1,4-¹³C₂]fumarate viafumarase is a sensitive indicator of cellular necrosis associated with acute kidney injury, tumor necrosis, and myocardial infarction. It was postulated that formation of HP [1,4-¹³C₂]malate in necrotic tissues was the result of easier access of fumarate to fumarase in damaged tissues than in normal ones. The ruptured plasma membranes of necrotic cells may allow small molecules such as fumarate, to rapidly enter cells or may facilitate the release of the enzyme into the interstitial space. Despite the potential for human applications, the poor solubility of both fumaric acid and sodium fumarate in conventional glassing solvents such as water, glycerol, and dimethyl sulfoxide (DMSO) is a primary limiting factor for achieving HP fumarate concentration necessary for successful in vivo studies.

No studies have reported HP malate or any other HP products from HP fumarate in healthy tissues. Blocking fumarate transport across cell membranes via the sodium-dependent dicarboxylate acid transporter (DCT) had no effect on the HP malate signal in necrotic tumor tissues, strongly suggesting that fumarate uptake and metabolism in intact cells is slow and does not contribute significantly to the HP malate signal. As a result, metabolic pathways such as malate-aspartate shuttle or gluconeogenesis that are directly associated fumarate have not been investigated using HP fumarate in normal tissues. However, the absence of products in normal tissues may be attributed to insufficient signal sensitivity.

In some examples, the current disclosure also encompasses use of the formulations to assess intracellular fumarate metabolism and its related pathways using the polarized formulation.

### IV. Terminology

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present disclosure or the appended claims.

Further, as the present disclosure is susceptible to aspects of many different forms, it is intended that the present disclosure be considered as an example of the principles of the present disclosure and not intended to limit the present disclosure to the specific aspects shown and described. Any one of the features of the present disclosure may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present disclosure may include a variety of combinations and/or integrations of the aspects described herein. Additionally, all aspects of the present disclosure, as described herein, are not essential for its practice. Likewise, other systems, methods, features, and advantages of the present disclosure will be, or become, apparent to one with skill in the art upon examination of the figures and the description. It is intended that all such additional systems, methods, features, and advantages be included within this description, be within the scope of the present disclosure, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to 1%, such as less than or equal to ±0.5%, such as less than or equal to ±0.2%, such as less than or equal to 0.1%, such as less than or equal to ±0.05%.

As used herein, the term “vitrification” is defined as the full or partial transformation of a substance into a glass.

As used herein, the term “formulation” is defined as a composition comprising a carboxylic acid, a glassing matrix, and a contrast agent. The formulation may include a surfactant/chelating agent. Examples of formulations include but are not limited to [1,4-¹³C₂]fumarate.

The terms “comprising,” “including” and “having” are used interchangeably in this disclosure. The terms “comprising,” “including” and “having” mean to include, but not necessarily be limited to the things so described.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

### Examples

The following examples are included to demonstrate preferred aspects of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the present disclosure.

### Preparation of Formulations

[1,4-¹³C₂]fumaric acid and meglumine were obtained from Sigma-Aldrich. OXO63 was obtained from GE Healthcare.

Two formulations were prepared and tested.

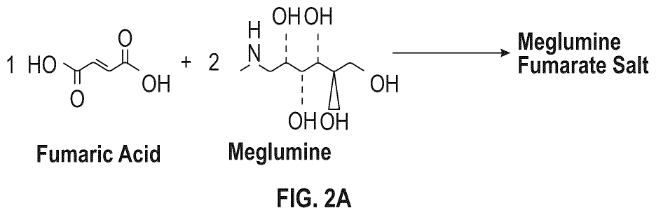

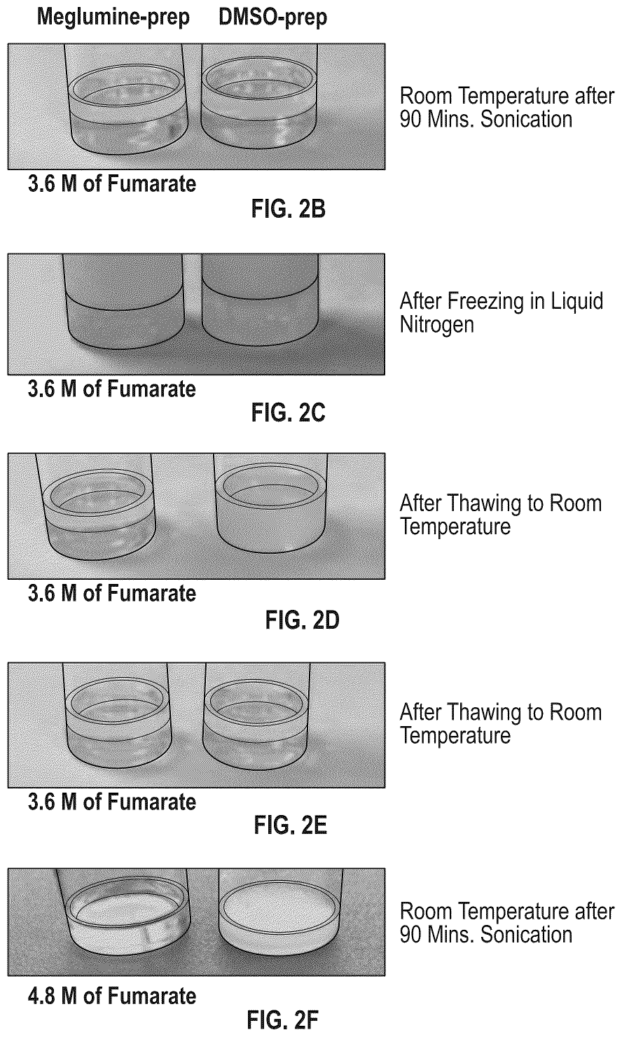

As shown in FIG. 2A, Formulation 1 of meglumine fumarate was prepared by mixing one equivalent of fumaric acid with two equivalents of meglumine free base in the presence of trace amount of water (approximately 6% by weight) followed by sonication for 90 minutes. 177 mg of [1,4-¹³C₂]fumaric acid (1.5 mmol) and 585 mg of meglumine (3 mmol) were added to 1.5-mL Eppendorf tube. 50 μL of water was added to the mixture. The mixture solution was vortexed and then sonicated in water bath (40° C.) for 90 minutes to generate the final concentrations of 3.6 M and 4.8 M with pH 7.0-7.4. The stock solution was stored in a −20° C. freezer until use.

Formulation 2 of conventional DMSO fumaric acid was prepared by mixing 152 mg of [1,4-¹³C₂]fumaric acid (1.29 mmol) and 264 mg of DMSO (3.38 mmol) were added to 1.5-mL Eppendorf tube. The mixture solution was vortexed and then sonicated in water bath (40° C.) for 90 minutes to generate the final concentrations of 3.6 M and 4.8 M with pH 7.0-7.4. The stock solution was stored in a −20° C. freezer until use.

Glassing properties of meglumine-mixed fumarate (Formulation 1) and conventional DMSO fumaric acid (Formulation 2) were compared at two concentrations: 3.6 M and 4.8 M. Both meglumine fumarate and conventional DMSO fumaric acid were prepared from natural abundance fumaric acid. At 3.6 M, meglumine fumarate and conventional DMSO fumaric acid solutions were transparent after 90 minutes of sonication in water bath at 40° C. (FIG. 2B). For testing the glassing matrix, the meglumine fumarate and conventional DMSO fumaric acid were rapidly frozen using liquid nitrogen. Both solutions formed a clear glass. (FIG. 2C). However, the fumaric acid solution in conventional DMSO became cloudy when warmed to room temperature. In contrast, the meglumine fumarate remained clear in solution after thawing (FIG. 2D). Maximum fumarate concentration was also higher with meglumine (4.8 M) than DMSO (3.6 M).

The solubility comparison between meglumine fumarate (formulation 1) and conventional DMSO fumaric acid (formulation 2) was performed at 4.8 M using natural abundance fumaric acid. The solution of the conventional DMSO fumaric acid (formulation 2) was cloudy after 90 minutes of sonication at room temperature whereas the meglumine fumarate (formulation 1) was clear and transparent (FIG. 2E). In formulation 2 (conventional DMSO fumaric acid), the fumaric acid crystallized out of solution at room temperature. The crystallized fumaric acid could not be re-dissolved by sonication at room temperature. The pH of the cloudy solution was 4. Notably, the solution of formulation 1 was transparent after thawing at room temperature which means that the signal can have much higher resolution as the formulation has not polarized, as with the conventional DMSO fumaric acid cloudy solution. The pH of the clear solution was 7.

A clear/transparent solution may be achieved with a concentration of 4.8 M meglumine [1,4-¹³C₂]fumarate in water in formulation 1. In contrast, 4.8 M of fumaric acid in conventional DMSO results in a cloudy solution in the conventional DMSO fumaric acid (formulation 2). a concentration of 3.6M.

As a result, in vitro and in vivo comparisons between meglumine fumarate (formulation 2) and conventional DMSO fumaric acid (formulation 2), both samples were prepared in 3.6 M. Hyperpolarization (HP) of Meglumine [1,4-¹³C₂]Fumarate (formulation 1)

3.6 M (or 4.8 M) meglumine [1,4-¹³C₂]fumarate (formulation 2), containing 15 mM trityl OXO63 (GE Healthcare, Waukesha, WI, USA), were polarized using a SPINlab DNP polarizer (GE Healthcare) that operates at ˜0.8 K in a 5 T magnet. 90 μL of the meglumine [1,4-¹³C₂]fumarate were placed in the sample vial and 17 mL of dissolution media (0.4 g/L (or 0.1 g/L) of ethylenediaminetetraacetic acid [EDTA] in water, pH 7.4) was added into the dissolution syringe of each research fluid path (GE Healthcare). The assembled research fluid path was loaded into the SPINlab. The sample was incrementally loaded with 17 steps without crystallization. After approximately 4 hours of polarization, the sample was dissolved with the heated dissolution media (130° C.), producing 6.5-7 mL of 50 mM (or 110 mM) hyperpolarized (HP) meglumine [1,4-¹³C₂]fumarate (pH 7.0). A high concentration of meglumine [1,4-¹³C₂]fumarate in formulation 1 results in up to about 110 mM after dissolution, which is equivalent to 220 mM of ¹³C.

Conventional Hyperpolarization of Sodium [1,4-¹³C₂]Fumarate Using DMSO as Glassing Matrix (Formulation 2)

3.6 M DMSO [1,4-¹³C₂]fumaric acid samples were prepared by dissolving [1,4-¹³C₂]fumaric acid in DMSO. 90 μL of 3.6 M DMSO [1,4-¹³C₂]fumaric acid containing 15 mM trityl OXO63 was placed in the sample vial for each fluid path with 17 mL of dissolution media (0.1-0.4 g/L of EDTA in water, pH 7.4) in the dissolution syringe. The assembled fluid path was loaded into the SPINlab and lowered using a one-step lowering option to avoid the crystallization of fumaric acid. The sample was loaded with one step, because subsequent steps resulted in crystallization. After approximately 4 hours of polarization, the sample was dissolved with the dissolution media and mixed with 0.5 mL of neutralizing media (0.72 M of NaOH, 0.4 M TRIS, and 0.1-0.4 g/L EDTA), producing 50 mM (or 80 mM) HP sodium [1,4-¹³C₂]fumarate (pH 7.0).

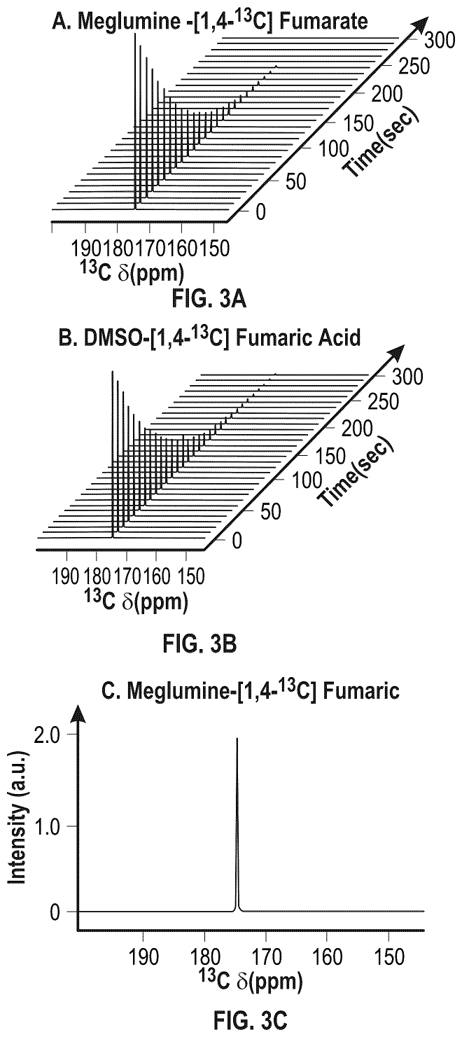

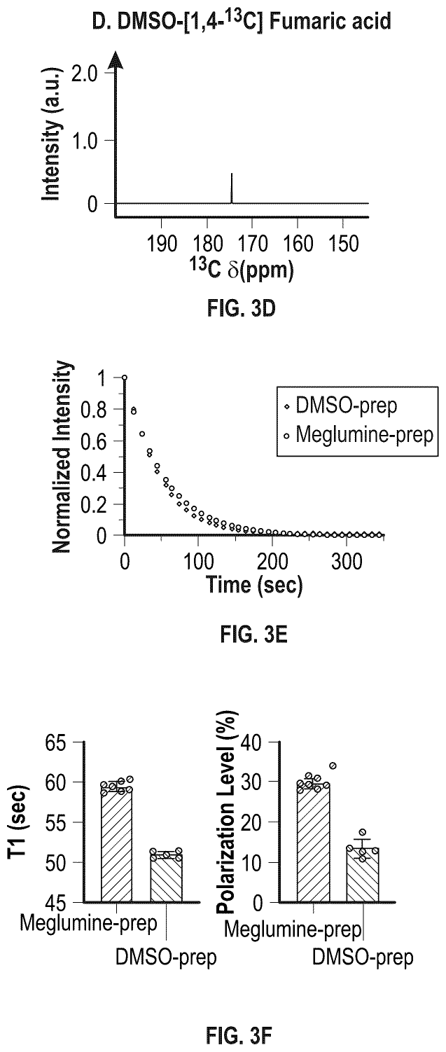

The T₁ of HP [1,4-¹³C₂]fumarate at 1 T was longer when the sample was prepared with meglumine (59.47±0.61 s, n=7; P=1.2×10⁻¹⁰) as compared to the DMSO-prepared fumarate sample (50.92±0.43 s, n=5). The T₁ of meglumine [1,4-¹³C₂]fumarate at 3 T (59.77±5.72 s, n=4) was comparable to the measurements at 1 T. The polarization level of meglumine [1,4-¹³C₂]fumarate was estimated as 29.42±1.31% (n=7), which is 2.18-fold of DMSO [1,4-¹³C₂]fumarate (13.48±2.40%, n=5; P=3.6×10⁻⁸). Since the doubly-labeled fumarate has a symmetrical molecular structure, the signal sensitivity is doubled, making the tangible polarization level of meglumine [1,4-¹³C₂]fumarate close to 60%. FIG. 3A shows time-resolved HP ¹³C normalized NMR spectra of meglumine [1,4-¹³C]fumarate. FIG. 3B shows time-resolved HP ¹³C normalized NMR spectra of conventional DMSO [1,4-¹³C]fumarate. FIG. 3C shows a representative first time-resolved HP ¹³C NMR spectra of meglumine [1,4-¹³C]fumarate. FIG. 3D shows a representative first time-resolved HP ¹³C normalized NMR spectra of conventional DMSO [1,4-¹³C]fumarate. FIG. 3E shows relaxation of the HP meglumine [1,4-¹³C]fumarate and conventional DMSO [1,4-¹³C]fumarate. FIG. 3F shows ¹³C polarization of meglumine [1,4-¹³C]fumarate and conventional DMSO [1,4-¹³C]fumarate.

T₁ values of meglumine fumarate were investigated at 1 T and 3 T. At 1 T, the T₁ of HP [1,4-¹³C₂]fumarate was nearly 20% longer when the sample was prepared with meglumine as compared to the conventional DMSO-fumaric acid samples that have been neutralized with NaOH. This is likely due to the quadrupolar relaxation effect of the sodium ions present in the conventional DMSO samples. The T₁ was maintained at a comparable level at 3 T. T₁ of [1,4-¹³C₂]fumarate at 11.7 T is 29 s using water as solvent. The longer T₁ at lower field reflects the effect of the dominant relaxation mechanism for the carboxylate, chemical shift anisotropy. The T₁ does not increase significantly at lower fields (<1 T) because additional relaxation mechanism becomes dominant: dipole-dipole relaxation from coupling to the nearby protons. Therefore, a T₁ of ˜60 s is expected in applications of meglumine [1,4-¹³C₂]fumarate in clinical imaging scanners, which typically employ field strengths of 1-3 T.

### In Vitro Measurements of Liquid-State Polarization and T₁

For each dissolution, ˜0.5 mL of HP [1,4-¹³C₂]fumarate was used to measure the polarization level and the T₁ relaxation time using a 1-T ¹³C NMR spectrometer (SpinSolve, Magritek, Malvern, PA, USA). Time-resolved ¹³C spectra were acquired every 10 seconds using 10° flip angle for ˜8 minutes until the HP signals disappeared. The liquid-state polarization level (P_{HP}) at the time of dissolution was estimated in vitro by comparing the first time-point of peak-integrated HP signal α_{HP} (50 mM) with the thermal equilibrium signal α_{HP} of neat [¹³C₆]benzene (12 M) using the equation 1.

$${P}_{HP} = {P}_{TH} \cdot ( \frac{{a}_{HP} \cdot sin {θ}_{TH} \cdot {c}_{TH}}{{a}_{TH} \cdot sin {θ}_{HP} \cdot {c}_{HP}} ) \cdot {e}^{\frac{{t}_{ds}}{{T}_{1}}} \qquad ( I )$$

Here, θ_{TH} and θ_{HP} are the radiofrequency (RF) flip angles (10° for both), used for sampling thermal and HP signals, respectively. The ¹³C concentration of the benzene sample, c_{TH}, and the fumarate sample, c_{HP}, are 72 M (12 M×6 carbons) and 0.1 M (0.05 M×2 carbons), respectively. The thermal polarization level (P_{TH}) was calculated for room temperature and 1 T. The signal decay during the transport time (t_{ds}) of the HP solution from the polarizer to the spectrometer was corrected by extrapolating the T₁ decay. The T₁ relaxation times of [1,4-¹³C₂]fumarate samples were measured by fitting the decay of the fumarate peak to a mono-exponential function after correcting the signal loss due to the RF sampling. The T₁'s of [1,4-¹³C₂]fumarate at 3 T were calculated from in vitro ¹³C spectra acquired using a dynamic ¹³C pulse-and-acquire pulse sequence (flip angle=5.625°, pulse width=32 μs, repetition time [TR]=3 s) with a clinical MRI scanner (Discovery 750w, GE Healthcare) and a ¹³C/¹H dual-tuned birdcage RF rat coil (Ø=80 mm, GE Healthcare).

Experimental Setup for In Vivo Imaging with HP [1,4-¹³C₂]Fumarate

Six male Wistar rats were used (body weight=338.8±19.2 g). Each rat was cannulated with a tail vein catheter under anesthesia (isoflurane level=2-3%), then placed in the ¹³C/¹H RF coil at the clinical 3 T system. After localization using a three-plane fast gradient echo (FGRE) sequence and B₀ shimming using a point-resolved spectroscopy (PRESS) in ¹H mode, 50-mM HP DMSO [1,2-¹³C₂]fumarate or HP meglumine [1,2-¹³C₂]fumarate was injected as a bolus through the catheter intravenously (0.625 mmol/kg body weight, up to 4.0 mL, injection rate=0.25 mL/s). ¹³C free-induction decay chemical shift imaging (FID CSI) was acquired 13 seconds after the start of the HP fumarate injection (field-of-view [FOV]=96 mm×96 mm, matrix size=16×16, slice thickness=15 mm, flip angle=10°, TR=75 ms, spectral width=5000 Hz, #spectral point=256, total scan time=19 s). For anatomical reference, T₂-weighted dual-echo ¹H fast spin echo (FSE; FOV=96 mm×96 mm, slice thickness=3 mm, flip angle=160°, TR=5,000 ms, echo times=12.33 ms/61.64 ms, echo train length=8, #slice=7) was acquired from the slab plane that was prescribed for ¹³C CSI. Three of the rats (344.7±26.0 g) were imaged with HP DMSO [1,2-¹³C₂]fumarate once. The other three rats (333±11.8 g) were imaged with HP meglumine [1,2-¹³C₂]fumarate twice with an interval of 2-4 days to compare fed and fasted (24 h) conditions. Two of the rats received additional bolus of HP meglumine [1,2-¹³C₂]fumarate for a dynamic ¹³C pulse-and-acquire scan (flip angle=10°, pulse width=1024 μs, spectral width=5000 Hz, #spectral point=1024, slice thickness=15 mm, TR=3 s) approximately 30 minutes after the first HP injection.

### Data Reconstruction and Analysis

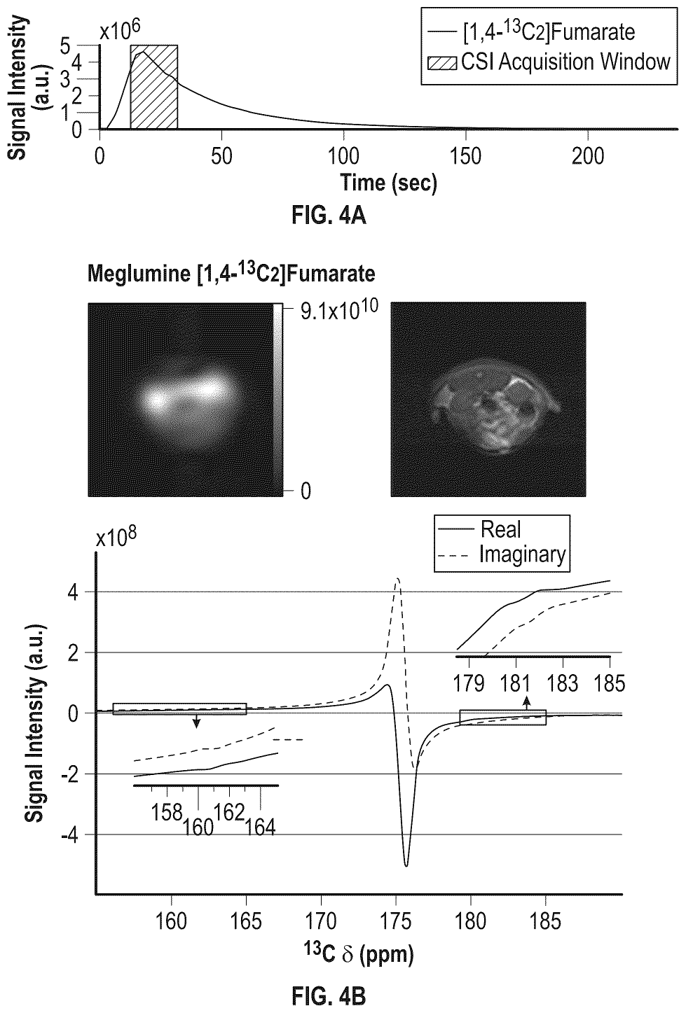

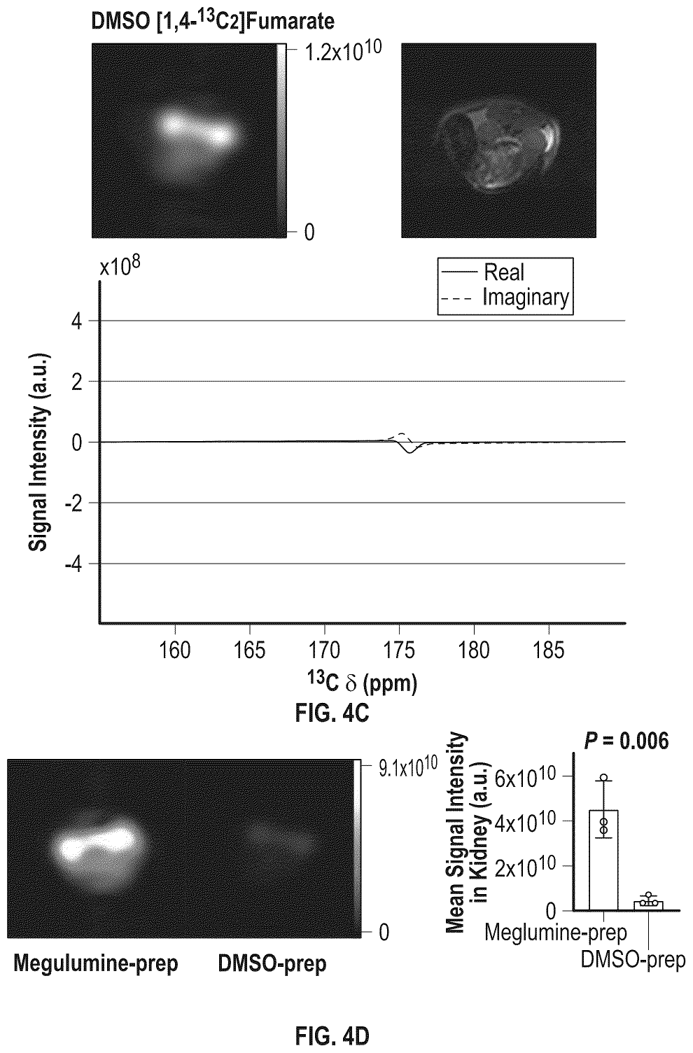

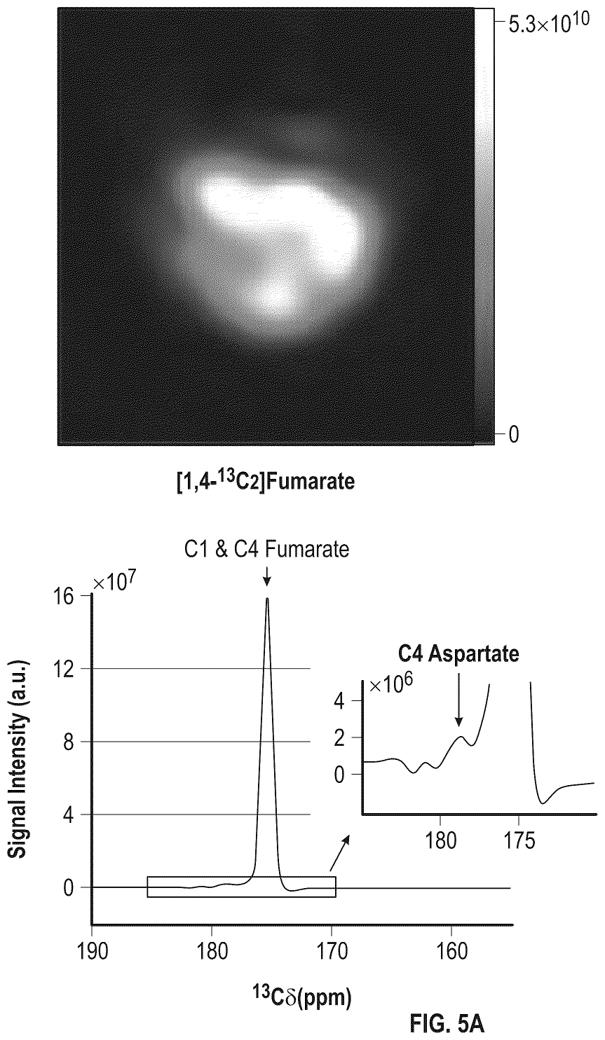

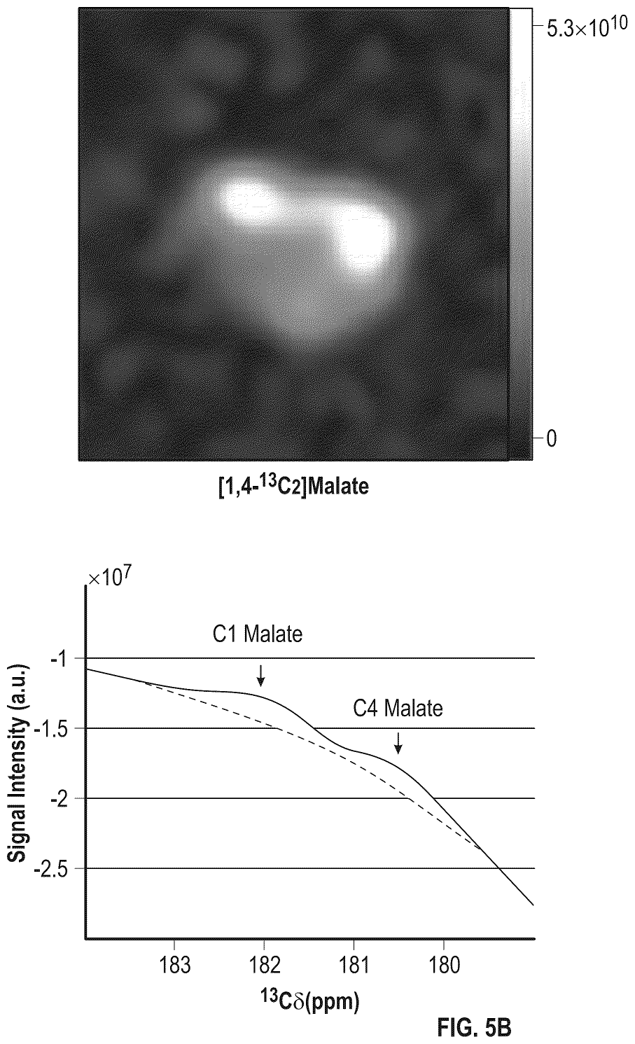

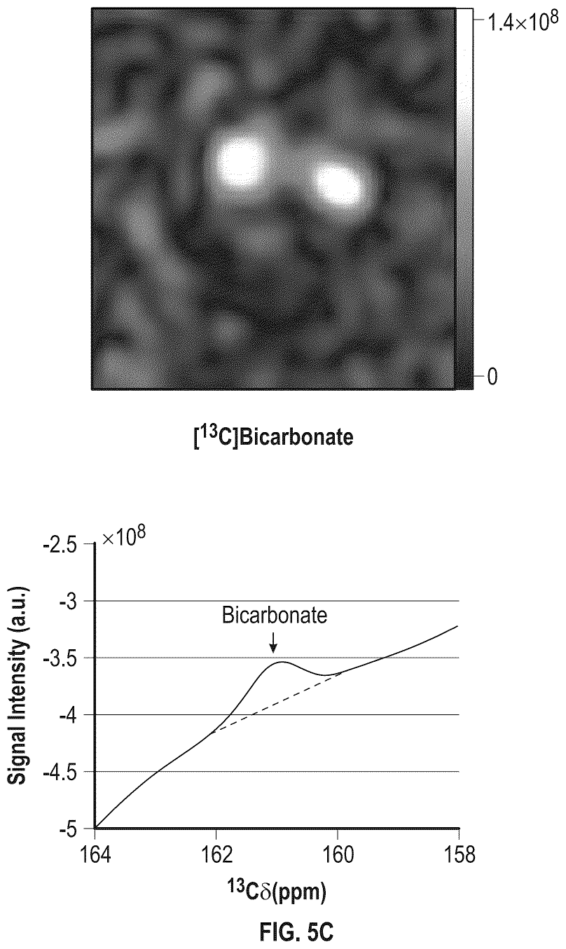

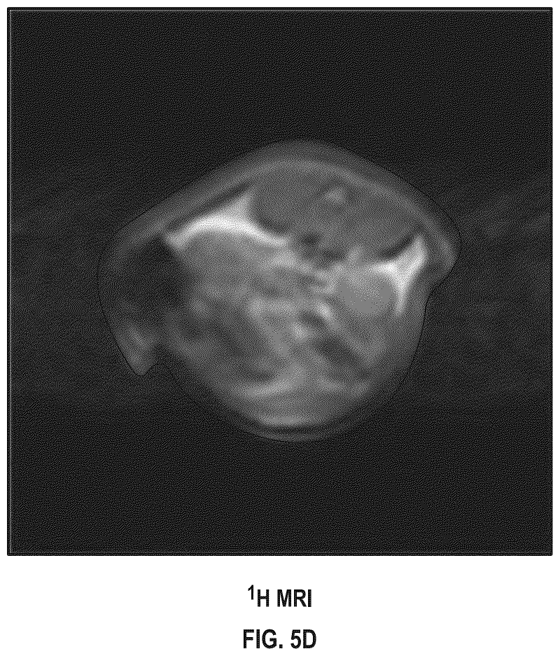

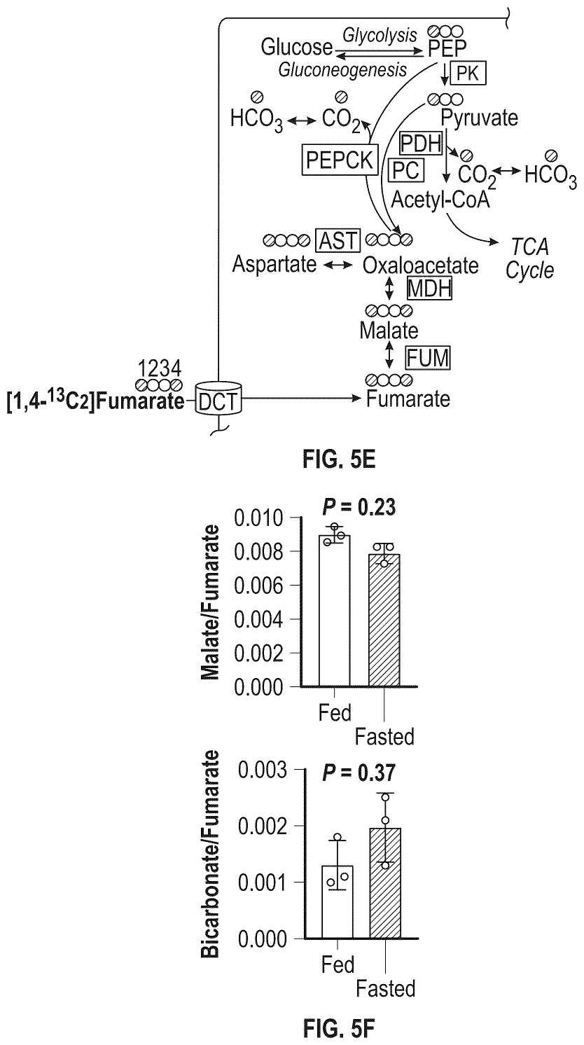

The ¹³C spectra acquired from the 1-T spectrometer were processed using Mnova (Mestrelab Research, A Coruña, Spain) for integrating HP [1,4-¹³C₂]fumarate signal. The k-space data of ¹³C CSI and dynamic ¹³C FID acquired from the 3 T scanner were reconstructed using MATLAB (Mathworks Inc., Natick, MA, USA) with 4-fold zero-padding in both spatial and frequency domains. Metabolite maps of [1,4-¹³C₂]fumarate, [1,4-¹³C₂]malate, and [¹³C]bicarbonate were generated by integrating the corresponding peaks in the absorption mode after 0th order phase correction in each voxel. For comparing the signal sensitivities of conventional HP DMSO [1,4-¹³C₂]fumarate and meglumine [1,4-¹³C₂]fumarate in the kidney, ROIs were created around the kidneys on the Trweighted ¹H MRI. All the presented in vivo ¹³C spectra (for example see FIGS. 4A-4D and FIGS. 5A-5F) were averaged over the ROIs and phase-corrected for both 0^{th} and 1^{st} orders. [1,4-¹³C₂]Malate and [13C]bicarbonate were quantified by integrating the peaks in the ROI-averaged spectra from both kidneys, then normalized by the averaged [1,4-¹³C₂]fumarate signal in the ROI. [1,4-¹³C₂]Aspartate was excluded from the analysis due to the difficulty of resolving the peaks from the large, spectrally adjacent [1,4-¹³C₂]fumarate.

### Statistical Evaluation

All results are reported as mean±standard deviation. For evaluating statistical significance of differences in T₁, polarization level, signal sensitivity, and SNR between DMSO-prepared [1,4-¹³C₂]fumarate and meglumine-prepared [1,4-¹³C₂]fumarate, a non-paired t-test was used (a=0.05, two-tailed). For evaluating the changes in [1,4-¹³C₂]malate and [¹³C]bicarbonate production due to fasting, each product in the kidney ROIs in fed and fasted conditions was compared using a paired t-test (a=0.05, two-tailed) after normalized by [1,4-¹³C₂]fumarate peak.

### Improvement of Sensitivity

The signal sensitivity of HP [1,4-¹³C₂]fumarate (175.4 ppm) was evaluated in healthy rodents in vivo at 3 T. FIG. 4A shows chemical shift imaging (CSI) was acquired 13 seconds after a bolus injection of HP [1,4-¹³C₂]fumarate. The HP fumarate signal intensity in the kidneys, averaged over the regions of interest (ROIs), was 4.49×10¹⁰±1.27×10¹⁰ with HP meglumine [1,4-¹³C₂]fumarate, which is more than 10-times higher than conventional HP DMSO [1,4-C₂]fumarate (4.42×10⁹±2.20×10⁹; P=0.006). Due to the dramatic improvement of in vivo signal sensitivity, additional peaks could be detected using meglumine [1,4-¹³C₂]fumarate (FIG. 4B). [1,4-¹³C₂]Fumarate images and averaged ¹³C spectra in the kidneys (solid line: real; dotted line: imaginary) using HP [1,4-¹³C₂]fumarate when prepared using meglumine and conventional DMSO are shown in FIG. 4B and FIG. 4C respectively. The kidney ROIs used for averaging are marked in the matching ¹H MRI (shaded red regions). FIG. 4D shows that as compared to conventional DMSO-prepared fumarate, the averaged signal sensitivity of in vivo [1,4-¹³C₂]fumarate increased by 10.2-fold for signal intensity in kidneys (P=0.006) when meglumine was used. As shown in FIG. 4B, due to the improved sensitivity, additional peaks could be detected using meglumine [1,4-¹³C₂]fumarate.

### In Vivo Detection of Gluconeogenic Products in Kidneys.

FIGS. 5A-F show in vivo detection of mitochondrial products in healthy rat kidneys using HP meglumine [1,4-¹³C₂]fumarate. FIG. 5A shows phase-corrected ¹³C spectra showing the in vivo detection of [1,4-¹³C₂]fumarate. FIG. 5B shows phase-corrected ¹³C spectra showing the in vivo detection of [1,4-¹³C₂]malate. FIG. 5C shows phase-corrected ¹³C spectra showing the in vivo detection of [¹³C]bicarbonate. The chemical shifts of the additional peaks were aligned with the resonances of [1-¹³C]malate (181.7 ppm), [4-¹³C]malate (180.3 ppm), [4-¹³C]aspartate (178.3 ppm), and [¹³C]bicarbonate (160.7 ppm). Metabolite maps of HP [1,4-¹³C₂]malate and HP [¹³C]bicarbonate could be generated by integrating the peaks in pure absorption mode after correcting the 0 order phase. As shown in FIG. 5A, [1,4-¹³C₂]Aspartate peaks were not resolvable from the large [1,4-¹³C₂]fumarate peak due to their adjacency. A cohort of rats were imaged twice under normal fed and 24-h fasted conditions to test whether they are sensitive to the nutritional condition. Both malate ([1,4-¹³C₂]malate-to-[1,4-¹³C₂]fumarate ratio; P=0.23) and bicarbonate ([¹³C]bicarbonate-to-[1,4-¹³C₂]fumarate ratio; P=0.37) did not show significant differences between the fed (malate/fumarate=0.0088±0.0005, bicarbonate/fumarate=0.0013±0.0004) and fasted states (malate/fumarate=0.0077±0.0006, bicarbonate/fumarate=0.0020±0.0006).

The signal intensity of HP meglumine [1,4-¹³C₂]fumarate was compared to that of conventional [1,4-¹³C₂]fumaric acid-DMSO samples in vivo at 3 T. The signal intensity of meglumine fumarate in the kidney ROIs is more than 10 times higher than that of conventional HP DMSO [1,4-¹³C₂]fumarate. Due to significant improvement of in vivo signal sensitivity of meglumine fumarate, metabolic intermediates other than [1,4-¹³C₂]malate could also be observed. The additional peaks at 160.7 ppm and 178.3 ppm were assigned to bicarbonate and C₄ of aspartate, respectively. The detection of aspartate and bicarbonate peaks using HP [1,4-C₂]fumarate either normal or necrotic kidney have not been reported. FIG. 5D shows a H MRI image of the corresponding images depicted in FIGS. 5A-C.

### Cellular Transport

Fumarate transport through the cell membrane via DCT is a relatively slow process, and although formation of malate is also observed in normal tissues, the malate/fumarate ratio is much higher (up to 300%) in regions of necrotic cells where fumarate has access to the enzyme. A previous study using ¹³C NMR on tissue extracts and superfusates reported that [U-13C4]fumarate (5 mM) metabolism to [U-¹³C₄]malate under oxygenated and hypoxic conditions suggesting uptake and metabolism of fumarate.

### Probing Gluconeogenic Pathway

Considering the slow cellular transport, the detection of malate, aspartate and bicarbonate after utilizing the HP meglumine [1,4-¹³C₂]fumarate was not expected and thus, surprising. Malate is converted to oxaloacetate in the TCA cycle and exported to the cytosol via the malate aspartate shuttle where it is converted to oxaloacetate. Alternatively, oxaloacetate can be produced from malate in the cytosol via malate dehydrogenase. In either case, the detection of HP [¹³C]bicarbonate indicates the capability of HP meglumine [1,4-¹³C₂]fumarate for probing gluconeogenic pathway. One of the labeled carbon from [1,4-¹³C₂]oxaloacetate is released as carbon dioxide via phosphoenolpyruvate carboxykinase (PEPCK) and detected as bicarbonate. The other labeled carbon follows phosphoenolpyruvate (PEP), which can be further metabolized to [1-¹³C]pyruvate then acetyl-CoA, releasing carbon dioxide via PDH as shown in FIG. 5E. However, the source of [¹³C]bicarbonate is more likely via PEPCK rather than PDH, considering that [1-¹³C]PEP, [1-¹³C]pyruvate and [1-¹³C]lactate were not detected. In vivo experiments to compare the signal of [4-¹³C]aspartate and [¹³C]bicarbonate in kidney in fasted and fed states showed higher bicarbonate/fumarate ratio in fasted animals compared to the fed group although this did not reach statistical significance. This rather persistent bicarbonate level is consistent with that renal gluconeogenesis is maintained stable until glycogen stored in the liver is depleted, which may require more than 24 hours. HP meglumine fumarate has unique potentials for investigating metabolic syndromes such as type 2 diabetes and chronic kidney diseases, which develop dysregulated gluconeogenesis. FIG. 5F shows bar graphs of the quantification of malate and bicarbonate production in rat kidneys under fed and fasted conditions.

### Feasibility of Clinical Translation.

Due to the extremely high solubility in polar protic solvents such as water (up to 1 g/mL), meglumine can increase the aqueous solubility of the formulation. Frozen, conventional DMSO-prepared fumaric acid samples became cloudy after thawing at room temperature, demonstrating its limitation in using which decreases exponentially with temperature decreases is a primary limiting factor to reach highly concentrated solutions of the HP substrate, which is necessary for human usage.

Meglumine is considered a safe, non-metabolizable expedient by the FDA. However, meglumine supplement was reported to have beneficial effect on metabolism. Analogously to the structurally related sorbitol, meglumine supplementation (18 mM in drinking water for at least 30 days) was shown to have a favorable effect on muscle function likely via elevating the expression of the AMPK-related kinase SNARK. However, at a single dose employed in HP ¹³C MRS studies, meglumine is unlikely to produce any metabolic effects.

What is claimed is:

1. A formulation for dynamic nuclear polarization (DNP)-nuclear magnetic resonance (NMR), the formulation comprising amino alcohol and a substrate;
wherein,
the amino alcohol includes meglumine;
the substrate comprises one or more of a free or ester form of an organic acid; and
the organic acid comprises C₃-C₆ monocarboxylic acid, C₃-C₆ dicarboxylic acid, C₃-C₆ hydroxy acid, C₃-C₆ α-keto-acid, amino acid, or a combination thereof.

2. The formulation of claim 1,
wherein,
the C₃-C₆ hydroxy acid comprises C₃-C₆ α-hydroxy acid, C₃-C₆ β-hydroxy acid, C₄-C₆ γ-hydroxy acid, or a combination thereof.

3. The formulation of claim 1, further comprising:
an aqueous medium.

4. The formulation of claim 1, further comprising:
a contrast agent.

5. The formulation of claim 4,
wherein,
the contrast agent includes a paramagnetic species such as free radicals, metal ions, nano particles, and/or a combination thereof.

6. The formulation of claim 1,
wherein,
the formulation is a substantially transparent solution at room temperature.

7. The formulation of claim 1,
wherein,
the C₃-C₆ monocarboxylic acid is butyric acid and the salt or ester is butyrate.

8. The formulation of claim 1,
wherein,
the C₃-C₆ dicarboxylic acid comprises fumaric acid, succinic acid, malic acid, oxaloacetic acid, or a combination thereof, and
the salt or ester is fumarate, succinate, maleate, oxaloacetate, and/or a combination thereof.

9. The formulation of claim 1,
wherein,
the C₃-C₆ hydroxy acid is lactic acid, and
the salt or ester is lactate, β-hydroxy butyric acid, malic acid, and/or a combination thereof.

10. The formulation of claim 1,
wherein,
the C₃-C₆ α-keto-acid is pyruvic acid, and
the salt or ester is pyruvate, oxaloacetic acid, α-ketoglutaric acid, and/or a combination thereof.

11. The formulation of claim 3,
wherein,
the combined amino alcohol and substrate has a concentration of at least about 3 M to about 5 M in the aqueous medium.

12. The formulation of claim 1,
wherein,
the combined amino alcohol and substrate has a concentration of at least 4.8 M in an aqueous medium.

13. A method of manufacturing a formulation for dynamic nuclear polarization (DNP)-nuclear magnetic resonance (NMR), the method comprising:
combining a substrate with meglumine to form a glassing matrix; and
adding a dissolution media to the glassing matrix to form a glassing matrix compound for DNP-NMR.

14. The method of claim 13,
wherein,
the dissolution media includes water with 0.1 g/L ethylenediaminetetraacetic acid (EDTA).

15. The method of claim 13,
wherein,
the glassing matrix compound for DNP-NMR is incrementally loaded into a polarizer using a multistep lowering procedure that includes more than two steps.

16. The method of claim 13,
wherein,
the glassing matrix compound for DNP-NMR is incrementally loaded into a polarizer using a multistep lowering procedure that includes between 10 and 20 steps.

17. The method of claim 16,
wherein,
the glassing matrix compound for DNP-NMR is incrementally loaded into a polarizer using a multistep lowering procedure that includes 14 steps.

18. The method of claim 13,
wherein,
the substrate includes fumarate, pyruvate, butyrate, amino acids, and/or lactate.

19. The method of claim 13,
wherein,
the combining the substrate with meglumine to form the glassing matrix further comprises combining a contrasting agent with the substrate and meglumine to form the glassing matrix, and
the contrasting agent includes a paramagnetic species such as free radicals, metal ions, nano particles, and/or a combination thereof.

20. A method of imaging a sample, the method comprising:
exposing the formulation of claim 1 to the sample; and
conducting dynamic nuclear polarization (DNP)-nuclear magnetic resonance (NMR) on the sample after exposure to the formulation.
